SEQUENCE LISTING (1 sequence)

SEQ ID NO: 1 - protein, 143 aa, Homo sapiens
  BINDING (27)..(40)
QDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

(19) United States
(12) Patent Application Publication
(10) Pub. No.: US 2025/0057920 A1
(43) Pub. Date: Feb. 20, 2025

(54) A MODIFIED PROTEIN OF INTERFERON GAMMA AND ITS USE THEREOF

(71) Applicants: CHIANG MAI UNIVERSITY, Chiang Mai (TH); SURANAREE UNIVERSITY OF TECHNOLOGY, Nakhon Ratchasima (TH)

(72) Inventors: Chatchai TAYAPIWATANA, Chiang Mai (TH); Umpa YASAMUT, Chiang Mai (TH); Montarop YAMABHAI, Nakhon Ratchasima (TH); Vannajan Sanghiran LEE, Kuala Lumpur (MY)

(21) Appl. No.: 18/249,932

(22) PCT Filed: Mar. 22, 2022
(86) PCT No.: PCT/TH2022/000011

§ 371 Date: Apr. 20, 2023

(51) Int. Cl. A61K 38/21; A61P 37/06; C12N 15/85

(52) U.S. Cl. CPC A61K 38/217; A61P 37/06; C12N 15/85

(57) ABSTRACT

A modified or bioengineered protein of a modified interferon gamma (IFN-γ) related to at least one modification. Preferably, the modified IFN-γ comprises at least one modification of an amino acid located at a position of 27 to 40 of a sequence as setting forth in SEQ ID NO. 1. The modified IFN-γ is capable of initiating cellular signaling towards an immune response upon being administered to a subject.

### TECHNICAL FIELD

The present disclosure relates to a modified or bioengineered protein bearing at least one modification. Particularly, the disclosed modified protein is a modified interferon gamma capable of initiating cellular signalling towards an immune response in a subject upon being administered to the subject at a sufficient amount substantially free from any adverse impact from the modifications bore.

### BACKGROUND

Interferon gamma (IFN-γ) is a type II interferon that plays pleiotropic roles in the innate and adaptive immune system [1]. It demonstrates anti-viral and anti-mycobacterial activity, antigen presentation by upregulation of major histocompatibility complex (MHC) molecules, anti-proliferative effects, and immunosuppression [2]. Structurally, IFN-γ is a homodimer, consisting of a non-covalent self-assembly in head-to-tail orientation. The helical regions A and B with their connecting loop, a histidine residue at position 111 (H111) in the F helix, and the flexible C terminus are important regions for receptor binding [3]. Ligand binding results in receptor oligomerization, with two α-receptor chains, IFN-γR1, bound to one IFN-γ homodimer, followed by recruitment of two β-receptor chains, IFN-γR2, to the complex inducing the expression of IFN-γ-stimulated genes [4, 5].

The presence of neutralizing anti-IFN-γ autoAbs is associated with adult-onset immunodeficiency (AOID) [6-10]. Patients lacking IFN-γ-mediated functions are susceptible to opportunistic infections, especially nontuberculous mycobacterial (NTM) infections. In 2016, Lin CH et al. identified an epitope recognized by anti-IFN-γ autoAbs using 30-mer non-overlapping synthetic peptides. The data illustrated the C-terminal region of IFN-γ (amino acid 121-131, SPAAKTGKRKR) as a sequential epitope recognized by the patient's autoAbs [11]. Recently, the neutralizing autoAb recognizing discontinuous epitope in patients with mycobacterial infection was identified (PCT publication no. WO 2018/202200 A1). Ku et al. also teaches a method for evaluating efficacy of recombinant human interferon gamma in regulating peripheral blood mononuclear cells in U.S. Pat. No. 10,273,278. Recently, the conformational epitopes recognized by other neutralizing anti-IFN-γ mAbs have been identified using human-bovine chimeric proteins. Accordingly, two major epitopes located at regions A and E were discovered [13]. Moreover, regions A and E-recognizing mAbs displayed various degrees of neutralizing activity. This finding confirmed that IFN-γ is composed of various conformational epitopes. However, epitopes recognized by autoAbs have not yet been fully investigated. More importantly, it is possible to derive a medicament based upon the information obtained relating to epitopes present on the IFN-γ.

### SUMMARY

The present disclosure aims to provide a bioengineered peptide or protein capable of forming interferon gamma, which bears modifications different from the wild type interferon gamma yet able to trigger cellular signalling towards an immune response in a subject upon administering to the subject at a pharmaceutically acceptable amount.

Further object of the present disclosure is to offer a modified protein incorporated with modifications allowing an interferon gamma formed thereto to escape interaction with at least one autoAbs, which prohibits initiation of the cellular signal towards an immune response.

Still, another object of the present disclosure is to offer a bioengineered interferon gamma carrying modification around a discontinuous epitope that one or more specific amino acids establishing the discontinuous epitope is modified to prevent at least one autoAbs present in a subject, who receiving the interferon gamma administrated, to react on the discontinuous epitope for blocking initiation of a cellular signal towards an immune response.

More object of the present disclosure is to cater a method to stimulate an immune response in a subject suffering from AOID by way of administering the abovementioned bioengineered peptide or modified interferon gamma protein.

According to several preferred embodiments, the present disclosure pertains to a modified or bioengineered protein of a modified interferon gamma (IFN-γ) comprising at least one modification of an amino acid located at a position of 27 to 40 of a sequence as setting forth in SEQ ID NO. 1. More preferably, the modified IFN-γ is capable of initiating cellular signalling towards an immune response upon being administered to a subject. The formed IFN-γ is resistant against at least one autoantibody capable of neutralizing a wild type IFN-γ at the discontinuous epitope.

In more embodiments, the modification alters a discontinuous epitope of the formed IFN-γ. Moreover, the modification comprises substituting at least one of amino acids located at position 27, position 29, and position 30.

For a number of embodiments, the at least one of amino acids located at position 27, position 29, and position 30 is substituted by alanine, aspartic acid, histidine, proline, or tryptophan.

Another aspect of the present disclosure relates to a method of initiating or stimulating a cellular signalling towards an immune response in a subject of adult-onset immunodeficiency (AOID) comprising the step of administering a plurality of bioengineered peptides and/or proteins. Preferably, the bioengineered peptide forms an interferon gamma protein comprising at least one modification of an amino acid located at a position of 27 to 40 of a sequence as setting forth in SEQ ID NO. 1.

For more embodiments of the disclosed method, the modification comprises substituting at least one of amino acids located at position 27, position 29, and position 30. More preferably, the at least one of amino acids located at position 27, position 29, and position 30 is substituted by alanine, aspartic acid, histidine, proline, or tryptophan in several embodiments.

Accordingly, in a number of embodiments of the disclosed method, the modification alters a discontinuous epitope of the formed IFN-γ rendering the formed IFN-γ resistant against at least one autoantibody capable of neutralizing a wild type IFN-γ at the discontinuous epitope.

More aspect of the present disclosure relates to a vector comprising a polynucleotide sequence being translatable to produce a protein forming an interferon comprising at least one modification of an amino acid located at a position of 27 to 40 of a sequence as setting forth in SEQ ID NO. 1. The modified IFN-γ is capable of initiating cellular signalling towards an immune response upon being administered to a subject.

### BRIEF DESCRIPTION OF THE DRAWINGS

FIG. 1 illustrates the query portion of human IFN-γ structure (PDB ID: 1FG9) where (A) is a single IFN-γ molecule, (B) shows the gap position of the position on IFN-γ (highlighted), (c) shows the side view of the homo-dimer arrangement of the IFN-γ molecule, and (D) shows the side view of the homo-dimer arrangement of the IFN-γ molecule;

FIG. 2 presents various rotational views of solvent accessible residues of ²⁷TLFLGILKNWKEES⁴⁰ in human IFN-γ molecule with the buried, exposed and intermediate residues were classified with respect to % SASA, <10%, >25%, and 10%-25% separately;

FIG. 3 illustrates further rotational view of intermolecular neighbours of ²⁷TLFLGILKNWKEES⁴⁰ in human IFN-γ molecule with the interactive and non-interactive residues on IFN-γ chain A and IFN-γ chain B being classified by the bond length between two chains of ≤5 and 5 Å respectively;

FIG. 4 is a graph showing solvent accessibility, intermolecular neighbours, and hydrogen bond properties of ²⁷TLFLGILKNWKEES⁴⁰ in IFN-γ with F29, L28, W36, L30 and N35 representing the hydrogen bonding;

FIG. 5 shows the predictive model query, where (A) is the query peptide, (B) is 1FG9_mt2g, (C) is 1FG9_mt0g, (D) is 1FG9, (E) is superimposed structure of the peptide and 1FG9, and (F) is superimposed structure of 1FG9_mt2g, 1FG9_mt0g, 1FG9, and their RMSD values;

FIG. 6 are graphs showing binding activity of anti-IFN-γ mAbs to the synthetic peptides including their neutralizing, where (A) presents the results of indirect ELISA for the binding of B27 or A9 mAbs to IFN-γ synthetic peptides from residues 25-42 and residues 128-143 and (B) presents the results relating to neutralizing capacity of B27 and A9 mAbs that the stimulation index was calculated from the expression of MHC class II after IFN-γ stimulation divided by that before stimulation;

FIG. 7 reveals the binding activity of B27 mAb to IFN-γ WT and mutants with (A) showing the amino acid sequence at positions 27-40 of IFN-γ WT and mutants, and (B) presenting western blot analysis of B27 mAb with respect to IFN-γ WT, T27A, T27AF29AL30A, and ΔT27-L33 that anti-6x His antibody was used to demonstrate the presence of each IFN-γ and the band intensity of IFN-γ obtained by B27 mAb was normalized by those detected using anti-6x His mAb while the number indicates relative level of each type of modified IFN-γ;

FIG. 8 illustrates amino acids arrangement of sequence listing, SEQ ID No. 1, of the wild type IFN-γ with the underlined portion denoting the section that can be subjected to the proposed modification to derive the disclosed modified IFN-γ; and

FIG. 9 illustrates the arrangements of various domains in vector pET-21a used in one embodiment of the present disclosure to translate or yield of the modified IFN-γ.

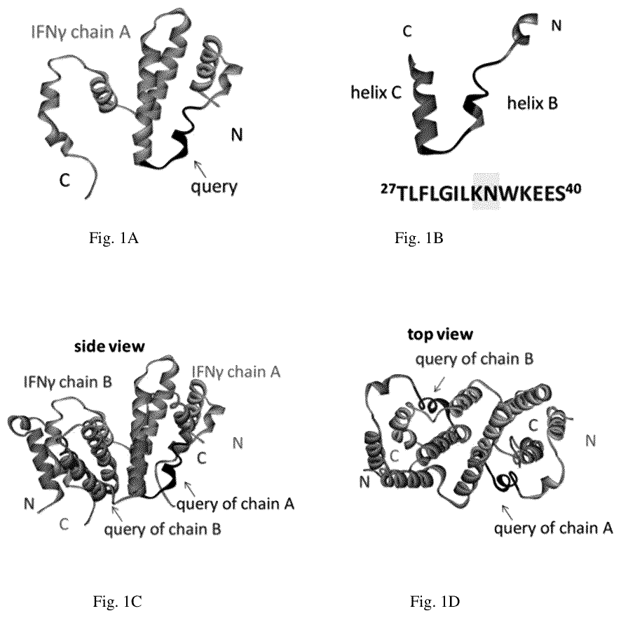

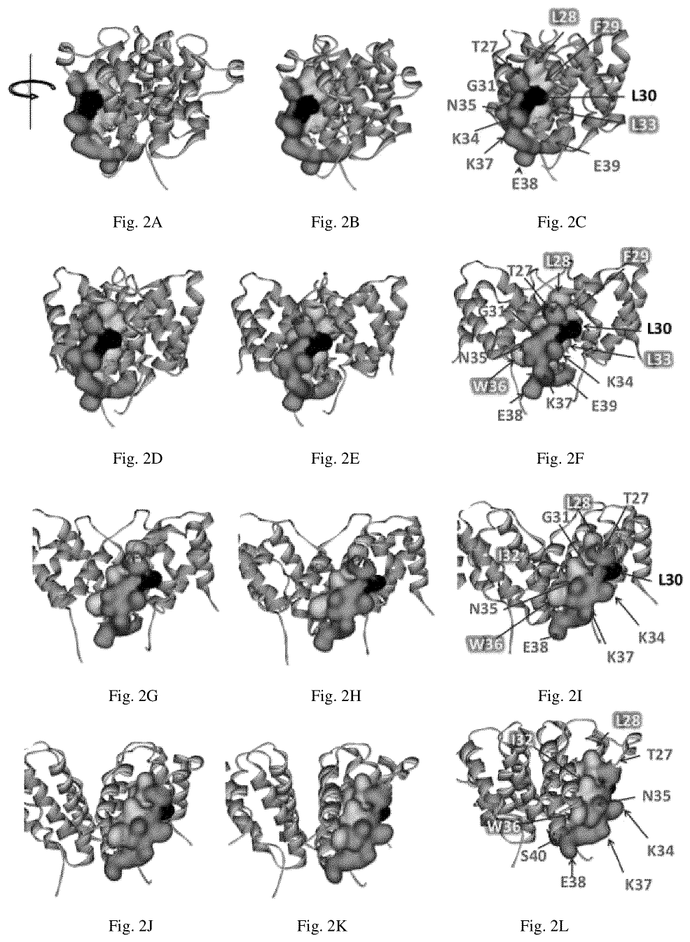

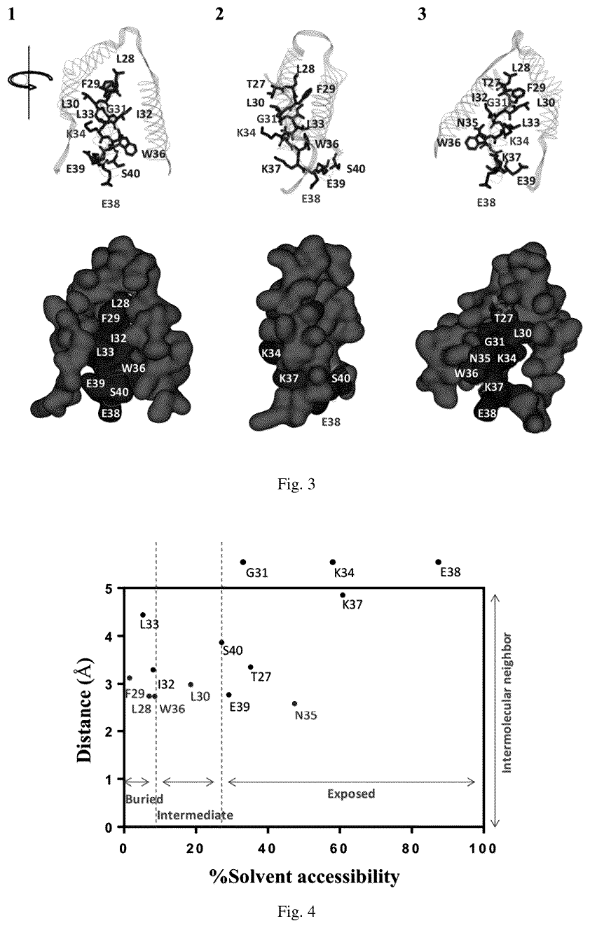

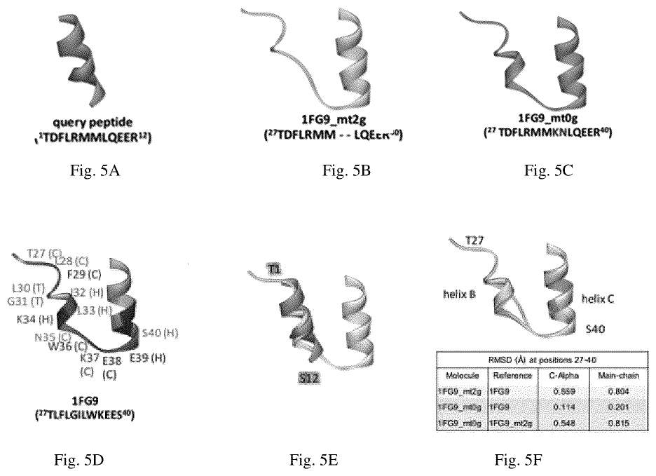

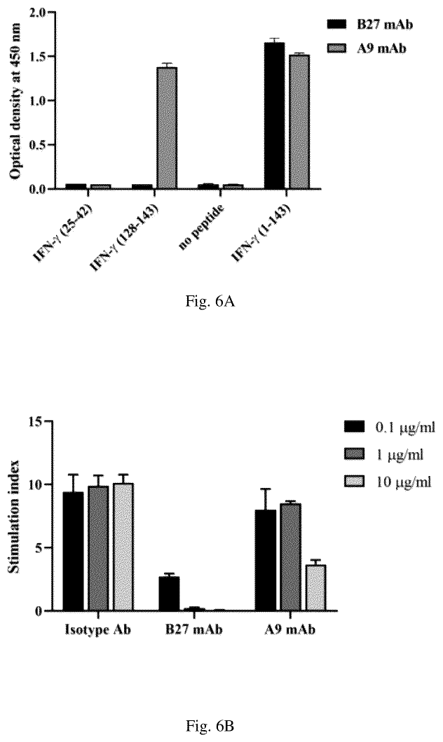

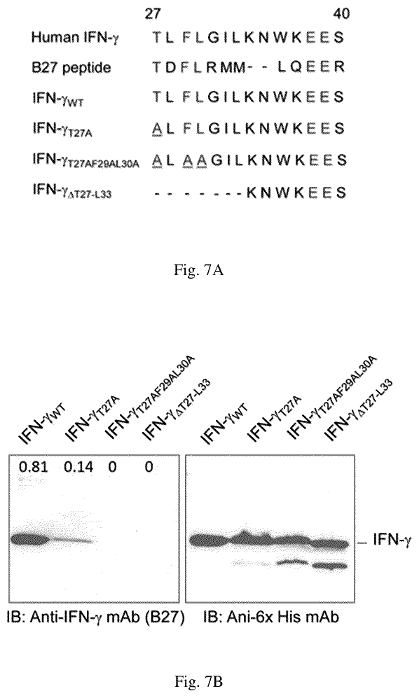

### DETAILED DESCRIPTION

Hereinafter, the disclosure shall be described according to the preferred embodiments and by referring to the accompanying description and drawings. However, it is to be understood that referring the description to the preferred embodiments of the disclosure and to the drawings is merely to facilitate discussion of the various disclosed embodiments and it is envisioned that those skilled in the art may devise various modifications without departing from the scope of the appended claim.

The terms “bioengineered protein”, “modified protein” and “bioengineered peptide” are used interchangeably in the present disclosure referring to a human-made, recombinant, or artificial protein being enabled to establish a modified IFN-γ molecule upon placing in a substantially in vivo environment. The modified IFN-γ has the capacity to initiate, trigger and/or activates an immune response in a subject receiving the modified IFN-γ in a pharmaceutically effective dosage.

The term “modification” used throughout the present disclosure shall generally refer to one or more changes introduced into a peptide sequence at the level of amino acids either biologically or chemically. The changes may involve addition, deletion, replacement, substitution and/or chemical alteration of one or more amino acids of the modified IFN-γ at one or more specific locations, compared to wild type IFN-γ, to confer the modified IFN-γ the preferred properties or traits. The modified peptide sequence shall preferably fold into a modified interested protein once it is subjected to a suitable environment.

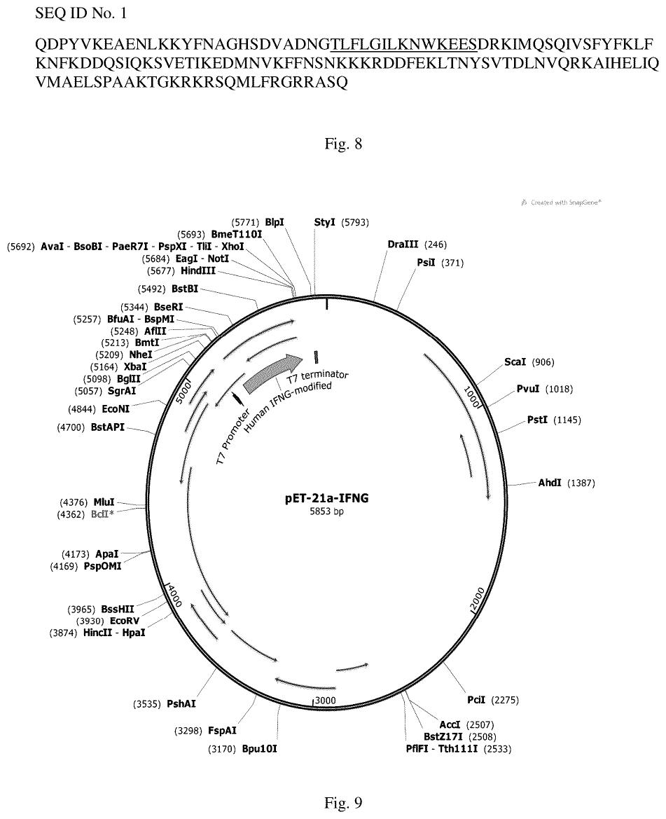

One aspect of the present disclosure associates to a bioengineered protein of a modified IFN or artificial IFN. The bioengineered protein or the proteins giving rise to the artificial IFN can be chemically synthesized or produced through any known biological methods. It is crucial that the modified IFN is preferably an IFN-γ. IFN-γ is a type II interferon serving the innate and adaptive immunity of a subject against infections. Natural or wild type IFN-γ is predominantly generated by natural killer cells and T-cells in a subject, particularly a mammal, in response to activation of innate immune response towards an infectious agent. For a number of embodiments, the bioengineered protein or bioengineered peptides comprises at least one modification of an amino acid located at a position of 27 to 40 of an amino acid sequence as setting forth in SEQ ID NO. 1. The amino acid arrangement, which is corresponding to a wild type of the IFN-γ, of SEQ ID NO. 1 is as shown in FIG. 8. It is important to note that the formed IFN-γ is capable of initiating cellular signalling towards an immune response, despite the modifications introduced thereto, upon being administered to a subject.

In accordance with several preferred embodiments, the modifications aim to alter a discontinuous epitope of the modified IFN-γ. It was discovered by the inventors of the present disclosure that the discontinuous epitope serves to bind onto IFN-γ receptor 1 (IFN-γR1) for inducing a cellular signal to trigger an immune response thereto. Nonetheless, the same discontinuous epitope can be targeted as well by at least one autoAbs commonly present in a subject suffering from adult-onset immunodeficiency (AOID). Binding of the autoAbs onto this particular discontinuous epitope results in failure of IFN-γ to interact with the IFN-γR1 consequently hindering initiation of the immune response. In view of that, the present disclosure proposes to create one or more modifications at the amino acids resided within the discontinuous epitope in a manner leading to the formed IFN-γ being resistant against at least one autoantibody capable of neutralizing a wild type IFN at the discontinuous epitope while the interaction with the IFN-γR1 remains intact or substantially unaffected. As mentioned in the foregoing description, the disclosed bioengineered proteins preferably comprise the modifications at the amino acids located at the position of 27 to 40 of SEQ ID NO. 1. Based upon the studies of the inventors, the amino acids located at these positions contribute to the establishment of the mentioned discontinuous epitope bindable towards both autoAbs and IFN-γR1. Thus, modification of one or more amino acids located at these positions can lead to a production of a modified IFN-γ incorporated with one or more desired features allowing the modified IFN-γ to substantially free from any disruption of the neutralizing autoAbs yet possessing the capacity to induce cellular signal towards an immune response upon administering the modified IFN-γ to the subject of AOID. The present disclosure further notes that the given range of amino acids of SEQ ID NO. 1 is, in fact, spanning across the several regions of the IFN-γ. In more specific, the regions involved are the region before helix B, the region within the helix B and the turning region between helix B and helix C.

Moreover, the present disclosure also uncovers that several key amino acids within the position of 27 to 40 play greater role for IFN-γ to react with the autoAbs and IFN-γR1. Specifically, amino acids located at position 27, 29 and 30 are found to be crucial in enabling binding of the autoAbs and IFN-γR1 onto the mentioned discontinuous epitope of the IFN-γ. Therefore, the present disclosure set out to institute the desired modification towards at least one of the amino acids located at these positions such that the changes made is minimal to retain the capacity of the modified IFN-γ to induce an immune response yet effective to avoid any substantial interaction with the autoAbs thereto. Preferably, the modification comprises substituting at least one of amino acids located at position 27, position 29, and position 30.

The type of modifications introduced is vital to determine efficiency of the formed modified IFN-γ considering that such modification aims to minimize the adverse impact towards the capacity of the modified IFN-γ in triggering the immune response. Pursuant to more preferred embodiments, the at least one of amino acids located at position 27, position 29, and position 30 is substituted by alanine. For more embodiments, it is possible as well to replace the at least one of amino acids located at position 27, position 29, and position 30 with aspartic acid, histidine, proline or tryptophan. It was discovered by the present disclosure that substituting the original amino acid type using one of these amino acids permits the modified protein to retain or even enhance the energy required for the discontinuous epitope to react with the receptor while effectively reducing the autoAbs to attach thereto. In a number of embodiments, the modifications may comprise substituting the amino acids located at position 27, position 29, and position 30 with any one of alanine, aspartic acid, histidine, proline or tryptophan.

Another aspect of the present disclosure refers to a method of initiating a cellular signalling towards an immune response in a subject of adult-onset immunodeficiency (AOID). A number of embodiments of the disclosed method comprises administering a plurality of bioengineered or modified proteins, which form a plurality of interferon each comprises at least one modification of an amino acid located at a position of 27 to 40 of a sequence as setting forth in SEQ ID NO. 1. To attain the object initiating cellular signalling and/or induction of an immune response, the plurality of bioengineered proteins shall be administered at a pharmaceutically effective amount throughout a given period of time. For more embodiments of the disclosed method may further comprise the step of preparing the bioengineered peptides prior to administering to the subject of AOID. For instance, the bioengineered proteins may be premixed with one or more reagent to reach an active form before being administered, possibly through an intravenous injection, to the subject. In some embodiments, the preparing step may involves diluting the bioengineered proteins to a preferred concentration before providing the bioengineered proteins to subject.

Likewise, the modification of the bioengineered proteins used in the disclosed method comprises substituting at least one of amino acids located at position 27, position 29, and position 30 of the SEQ ID NO. 1. As mentioned in the setting forth, the amino acids located at the position 27, position 29, and position 30 are found to be the key amino acids enabling function of the IFN-γ in prompting a cascade of cellular signal for an immune response via the discontinuous epitope, which these key amino acids are resided. According to the inventors of the present disclosure, the T27 amino acid at the position 27 of the SEQ ID NO. 1 is approximate to the AB connecting loop and it is essential for enabling the IFN-γ and IFN-γR1 interaction. As such, any modifications performed towards T27 and/or any other amino acids located across position 27 to 40 can give rise to diminished interaction of the modified IFN-γ in relation to the IFN-γR1 or the autoAbs. The present disclosure unveils that the modified IFN-γ having at least one of amino acids located at position 27, position 29, and position 30 substituted by any one of by alanine, aspartic acid, histidine, proline and tryptophan only leads to reduced reactivity with regards to autoAbs, which target the discontinuous epitope containing these amino acids, while the interaction with IFN-γR1 appears corresponding or almost corresponding to wild type IFN-γ. By way of administering the bioengineered proteins bearing these modifications, the disclosed method offers a way to induce a desired immune response generally absence in the subjects of AOID having autoAbs targeting the wild type IFN-γ. More specifically, the modification alters a discontinuous epitope of the formed IFN or the formed bioengineered IFN-γ rendering the formed IFN resistant against at least one autoantibody capable of neutralizing a wild type IFN at the discontinuous epitope.

Further aspect of the present disclosure may cover a vector comprising a polynucleotide sequence being translatable in a compatible cell to produce a bioengineered protein of a modified interferon comprising at least one modification of an amino acid located at a position of 27 to 40 of a sequence as setting forth in SEQ ID NO. 1. Preferably, the modified or formed IFN-γ is capable of initiating cellular signalling towards an immune response upon being administered to a subject. In accordance with several preferred embodiments of the disclosed vector, the compatible cells are BL21(DE3) cells. The compatible cells can be configured to harbour the disclosed vector through transformation or the like process. The compatible cells transformed with the disclosed vector can be cultured in a suitable medium for a period of time such that the transformed cells may express the bioengineered proteins.

In some embodiments, the commercial vector applicable to be incorporated with the DNA sequence of modified DNA sequence is pET-21a or pQE-10. FIG. 8 presents one embodiment of the disclosed vector based upon pET-21a. Also, the compatible cells usable to house the disclosed vector for expression of the modified proteins may be varied according to the type of vector used.

In order to yield the bioengineered protein of the modified IFN-γ, the modification introduced to the SEQ ID NO. 1 carried by the disclosed vector comprises substituting at least one of amino acids located at position 27, position 29, and position 30. More preferably, the at least one of amino acids located at position 27, position 29, and position 30 is substituted by alanine, aspartic acid, histidine, proline, and/or tryptophan.

The following example is intended to further illustrate the disclosure, without any intent for the disclosure to be limited to the specific embodiments described therein.

### EXAMPLE 1

Affinity selection or biopanning process against 12-mer phage display random peptide library (SUT12) was performed, as described previously [15]. Briefly, three rounds of biopanning were performed by reducing the amount of anti-IFN-γ mAbs (clone B27, ImmunoTools, Friesoythe, Germany), from 10, 5, to 2 μg, in each consecutive round of affinity selection. After the first round of biopanning, the eluted phage was amplified overnight. No phage amplification was performed after the second round. Individual phage clones obtained after the third round of biopanning were amplified. Their binding activity against B27 mAb was detected by phage ELISA, as reported previously [16]. To determine the amino acid sequences recognized by B27 mAb in bound phage, phagemid from positive phage clones were prepared. The DNA sequences were determined by automated DNA sequencing services using the −96gII primer (5′-CCC TCA TAG TTA GCG TAA CG-3′). The amino acid sequences were analyzed using SnapGene software.

### EXAMPLE 2

The query peptide sequence, TDFLRMMLQEER, was aligned with the full-length sequence of human IFN-γ using pairwise alignment algorithm in BioEdit. Local alignment, known as ‘allow end to slide’ in BioEdit, with a BLOSUM62 similarity matrix, gap initiation penalty of 8, and gap extension penalty of 2, was used for the calculation of identity and similarity.

Specific amino acid residues, related to the query sequence, of the 3D structure of human IFN-γ (PDB ID: 1FG9) were analyzed for their interactive properties, such as water accessibility, intermolecular neighbors, and intermolecular hydrogen bonding. First, the homodimeric structure of IFN-γ was selected for calculation of the solvent-accessible surface area (SASA) using an enhanced grid-based numerical algorithm with 240 grid points per atom and a 1.4 Å probe radius. Residues with SASA <10% were defined as buried residues, whereas the ones with value beyond the threshold of 25% were called exposed residues. Second, intermolecular neighbors were considered if there were at least two atoms, one belonging to IFN-γ (chain A) and another to IFN-γ (chain B), having a bond distance <5 Å. Finally, intermolecular bond with distance within 3.5 Å, and angles XDA and DAY within 0 to 180 degrees were used to identify an intermolecular hydrogen bond.

### EXAMPLE 3

Comparative and predictive 3D structures of the query were built using 3 different structures, one peptide and two mutant IFN-γ molecules. The peptide structure consisted of 12 residues, and its sequence was identical to the query, TDFLRMMLQEER. For mutant forms, the query structures were constructed together with other parts of human IFN-γ. Using the same template structure (PDB ID: 1FG9), the protein sequences were found being similar to that in the full-length human IFN-γ, except in positions 27-40, which were substituted by 27TDFLRMM--LQEER40 and 27TDFLRMMKNLQEER40, referred to as 1FG9_mt2g (127 residues) and 1FG9_0g (129 residues), respectively. K34 and N35 of 1FG9_mt0g were copied from human IFN-γ. Different target sequences were employed to search for the template, with BLAST and HHblits using the SWISS-MODEL server, in order to obtain the target-template alignment. The models were built based on target-1FG9. A alignment using ProMod3.

### EXAMPLE 4

The 3D structure of human IFN-γ (PDB ID: 1FG9) was downloaded from Protein Data Bank (PDB) [22]. After removal of water molecules and non-protein molecules, missing atoms were added and the initial structure was optimized to remove steric clashes using AMBER forcefield in HyperChem 7.5 software package by short minimizations until Root Mean Squared (RMS) gradient tolerance of 0.1000 (kcal/ (Å mol)). The amino acid at the position T27 was mutated with UCSF Chimera and followed by short minizations in similar manner. The interaction energy (IE) analysis was calculated from IE =EAB−EA−EB where A, B indicated each residues fragment in forming AB complex. Interaction with the receptor was explored for amino acids in 5 Å vicinity from IFN-γ. In addition, online prediction tool (PRODIGY, http://milou.science.uu.nl/services/PRODIGY) was used for analysis a contact-based predictor of binding affinity in protein-protein complex [24].

### EXAMPLE 5

Site-directed mutagenesis was conducted to generate plasmid pET21a IFN-γ T27A using the QuickChange® lightning multisite-directed mutagenesis kit (Stratagene, La Jolla, CA), as per the manufacturer's instruction. IFN-γ T27A was amplified by PCR using the plasmid pET21a IFN-γ as a template. The primers used were 5′attcttcaaaatgcctaagaaaagcgctccattatccgctacatctgaatg-3′ and 5′-cattcagatgtagcggataatggagcgcttttcttagcattttgaagaat-3′. The PCR reaction was performed with an initial denaturation step at 95° C. for 2 min, followed by 18 cycles of denaturation at 95° C. for 20 s, annealing at 60° C. for 10 s, and extension at 68° C. for 75 s, and a final extension at 68° C. for 5 min. The PCR product was digested with DpnI at 37° C. for 5 min, to eliminate any methylated parental DNA template, and transformed into competent E. coli strain XL1-Blue. The correct mutant clone was verified by digestion with HindIII and Nhel. Colony PCR was subsequently performed with AmpMaster™ Taq Master Mix, GeneAll Biotechnologies, using the primers 5′-gaggaggagaagcttttagtgatggtggtgatggtgaccagaagactgggatgctcttcg-3′ and 5′-gaggaggaggctagcatgcaggacccatatgtaaaagaagcagaaaaccttaagaaa-3′. PCR reaction was conducted with an initial denaturation step at 95° C. for 2 min, followed by 30 cycles of denaturation at 95° C. for 30 s, annealing at 55° C. for 30 s, and extension at 68° C. for 1 min, and a final extension at 68° C. for 5 min. Finally, the plasmid pET21a IFN-γ mutant clone with T27A was extracted from E. coli strain XL1-Blue using the QuickGene-Mini80 kit to perform DNA sequencing.

### EXAMPLE 6

The cDNA sequence of IFN-γ mutants, including IFN-γT27AF29AL30A or IFN-γΔT27-L33from pUC57 plasmid, was subcloned into pET21a IFN-γ using Nhel and Bsp119I restriction enzymes. After ligation, the pET21a plasmids containing mutated IFN-γ sequence were transformed into competent E. coli strain XL1-Blue. Colony PCR was performed using the primers 5′-gaggaggagaagcttttagtgatggtggtgatggtgaccagaagactgggatgctcttcg-3′ and 5′-gaggaggaggctagcatgcaggacccatatgtaaaagaagcagaaaaccttaagaaa-3′. The correct mutant clone was verified by digestion with HindIII and Nhel. The plasmid pET21a IFN-γ mutant clones with IFN-γT27AF29AL30A or IFN-γΔT27-L33 were extracted from E. coli strain XL1-Blue using the QuickGene-Mini80 kit to perform DNA sequencing.

### EXAMPLE 7

The plasmid encoding IFN-γ wild-type (WT) or mutants was transformed into BL21 (DE3) competent cells to produce recombinant IFN-γ protein (rIFN-γ). The cells were grown in 5 ml of super broth (SB) medium at 37° C. overnight and subsequently inoculated in 500 ml of SB medium, containing 1% glucose and 100 μg/ml ampicillin, at 37° C. Protein expression was induced by adding 1 mM IPTG when the optical density of the culture at 600 nm (OD600 nm) reached 0.8-1.0, and the culture was continued thereafter for 16 h at 30° C. The induced cells expressing rIFN-γ were washed with phosphate buffered saline (PBS), lysed by freeze-thaw with 5-min sonication thrice, followed by centrifugation at 15,000 ×g, 4° C. for 30 min. The cell pellets were collected for western blotting.

### EXAMPLE 8

To verify whether anti-IFN-γ mAb (clone B27) binds to IFN-γ position 27-40, indirect ELISA was performed. Microtiter plates were coated with 50 μl of streptavidin (2 μg/ml) in bicarbonate buffer (pH 9.6) per well and incubated overnight at 4° C. in a moist chamber. The other steps were performed at 37° C. in a humidified chamber. The coated wells were washed four times with 0.05% Tween 20 in PBS. Peptide 25NGTLFLGILKNWKEESDR42 labelled with biotin (2 μg/ml) was added and incubated for 1 h. After washing, non-specific binding was blocked with a blocking solution (2% skim milk in PBS) for 1 h, and 50 μl of B27 mAb (0.5 μg/ml) was added and incubated for 1 h. After washing four times, 50 μl of HRP-conjugated goat anti-mouse immunoglobulin (dilution 1:3,000) was added and incubated for 1 h. The reactions were developed with TMB substrate and stopped with 1 N HCl. Absorbance was measured at 450 nm with an ELISA reader. In this experiment, anti-IFN-γ mAb (clone A9, Santa Cruz Biotechnology, CA, USA), which recognizes IFN-γ at positions 125-143, was used as the positive control of detection system.

### EXAMPLE 9

Purified IFN-γ WT or pellet of BL21 (DE3) cells expressing IFN-γ mutants were subjected to SDS-PAGE under reducing conditions and then transferred to nitrocellulose membrane. The membrane was blocked with 5% skimmed milk in PBS overnight at 4° C. Anti-IFN-γ mAb, clone B27 (1 μg/ml), or anti-6x His antibody (0.5 μg/ml) were separately added and incubated with the membrane for 1 h at room temperature with shaking. After washing, the membranes were incubated with an HRP-conjugated goat anti-mouse immunoglobulin antibody (dilution 1:3000 in 2% skimmed milk in PBS) for 1 h. The membranes were washed, and bands were enhanced using Supersignal™ West Pico Chemiluminescence Substrate (Thermo Fisher Scientific); the protein bands were visualized under a ChemiDoc™ MP imaging system (Bio-Rad).

### EXAMPLE 10

To determine the neutralizing activity of B27 and A9 mAbs, a cell-based assay was performed. Briefly, 10 ng/ml of rIFN-γ WT was incubated with mAb at 0.1, 1, or 10 μg/ml for 1 h. The mixture was subsequently incubated with THP-1 cells (4×10⁵ cells) at 37° C. in 5% CO₂ incubator. After 24 h, cells were harvested to detect MHC class II surface expression by flow cytometry. Cells were washed thrice with PBS and blocked with 10% AB serum in PBS for 30 min. For MHC class II staining, 50 μl of FITC-conjugated anti-human HLA-DR and -DP (clone HL-38) or isotype-matched control was added to FITC-conjugated mouse IgG2a (ImmunoTools, Friesoythe, Germany) and incubated for 30 min on ice. After washing, cells were resuspended in 1% paraformaldehyde-PBS. Data were collected with BD Accuri™ C6 Plus Flow Cytometer (BD Bioscience).

### EXAMPLE 11

The presence of anti-IFN-γ autoAbs is tightly associated with severe NTM and intracellular pathogen infections in patients with AOID [6-10]. Disruption of IFN-γ-mediated signalling by neutralizing autoAbs results in immune defects [20]. The IFN-γ-IFN-γR1 interaction is the initial step for IFN-γ-mediated signalling. The regions of IFN-γ involved in receptor binding have been reported to include the loop connecting the A and B helices (residues 18-26), the helix F, and the C-terminal region [3]. Recently, the autoAb against C-terminal epitope was found in patients with mycobacterial infection [11], which indicated that autoAbs block receptor binding, inhibit IFN-γ signalling, and result in the development of immunodeficiency syndrome. Interestingly, the C-terminal peptide was recognized by autoAbs in only 40% of the 63 AOID cases reported previously [21]. Such findings indicated that either linear or conformational epitope stimulate the production of neutralizing anti-IFN-γ autoAbs. To understand the mechanisms underlying the pathogenesis of AOID better, anti-IFN-γ autoAbs from patients should be characterized. In a previous study conducted by the inventors, the autoAbs of patients with AOID recognized the same epitope as B27 mAb [12]. Since B27 mAb did not recognize the C-terminal epitope identified by Lin CH et al. [11], the epitope for a particular autoAb in patients with AOID was investigated further. In the present disclosure, a phage display random peptide library was selected to identify the B27 epitope. Although six phage clones were sequenced, only one peptide “TDFLRMMLQEER” was retrieved, which suggested that B27 mAb has favorable binding affinity to this particular sequence. The obtained sequence was further analyzed using BLAST and HHblits through SWISS-MODEL server. The predicted sequence relying on IFN-γ (PDB ID: 1FG9) was 27TLFLGILKNWKEES40, located just before helix B, in helix B, and in the turn between helices B and C. However, the binding of B27 mAb to this sequence was negative in ELISA, which suggested that B27 epitope possibly relies on the discontinuous structure of IFN-γ.

### EXAMPLE 12

To further verify the implication of this region in IFN-γ bioactivity, computer-based analysis was performed. From PRODIGY analysis (Table 1), T27 was identified as the key amino acid interacting with IFN-γR1 at Y49, G50, and N79. The T27 residue was close to the AB connecting loop (position 18-26) that was essential for receptor interaction. The data implied that the autoAb against this region affects the IFN-γ/IFN-γR1 interaction. Results from western blotting illustrated that the binding activity of B27 mAb to T27A was significantly decreased compared to that in the wild type. An epitope of B27 mAb was further investigated using IFN-γ mutants in which amino acids T27, F29, and L30 were substituted with alanine and seven amino acids from T27 to L33 were deleted. Binding of B27 mAbs to the triple mutant and T27-L33 deletion mutant was dramatically reduced, and deletion of the sequence affected the loop structure in IFN-γ. The data confirmed that B27 mAb interacted favourably with the loop structure. Deletion of T27-L33 was found to affect the interaction energy, although T27AF29AL30A gave results similar to IFN-γ wild-type. The findings overall supported the idea that neutralizing antibody specific to B27 mAb-recognized epitope is crucial for the inhibition of cellular signalling of IFN-γ.

TABLE 1

| IFNG | Receptor | IE (kcal/mol) |
|---|---|---|
| THR27 | | |
| Chain B | Chain D | 276.80 |
| ASN25-ALA32 | TYR49 GLY50 ASN79 | 11.74 |
| THR27 | TYR49 GLY50 ASN79 | 4.29 |
| THR27 | TYR49 | 3.02 |
| THR27 | GLY50 | 1.07 |
| THR27 | ASN79 | 0.19 |
| ALA27 | | |
| Chain B | Chain D | 294.09 |
| ASN25-ALA32 | TYR49 GLY50 ASN79 | 11.18 |
| ALA27 | TYR49 GLY50 ASN79 | 3.30 |
| ALA27 | TYR49 | 2.27 |
| ALA27 | GLY50 | 0.96 |
| ALA27 | ASN79 | 0.08 |

According to the formerly identified C-terminal epitope [11], the neutralizing efficiency of A9 mAb, specific to KTGKRKRSQMLFRGRRASQ, and B27 mAb was compared. Results revealed that B27 mAb neutralized IFN-γ activity much more efficiently than A9 mAb at the same concentration. The C-terminal region is not visible in the crystal structure (PDB ID: 1FG9) due to its flexibility. A previous report had demonstrated that C-terminal sequence of IFN-γ is a heparin sulphate-binding domain [23]. Heparan binding to IFN-γ resulted in the interference of IFN-γ/IFN-γR1 interaction. Although this region seemed to participate in receptor binding, truncated IFN-γ with deletion of C-terminal portion did not significantly alter the binding affinity of IFN-γ to IFN-γR1 [23]. Consequently, we suggested that A9 mAb interacts with the C-terminal epitope beyond the primary interaction of IFN-γ/IFN-γR1 and partially hinders the occurrence of 2:2:2 IFNγ-IFNyR1-IFNγR2 complex. In contrast, B27 mAb prevents the initial interaction of IFN-γ with IFN-γR1, which is a crucial step for cellular signalling. The present disclosure is in view that the presence of neutralizing antibody against B27 epitope is involved in IFN-γ signalling deficiency. The evidence might mimic the pathogenesis in patients with AOID.

Apart from the autoAbs against IFN-γ, neutralizing Abs are commonly found in patients

with anti-cytokine autoAb disease (ACAD) [24]. There are several approaches for identifying the epitopes recognized by anti-cytokine autoAb. For anti-granulocyte macrophage colony-stimulating factor (GM-CSF) autoAbs in patients with IPAP, the neutralizing epitopes were characterized by the generation of mAbs against GM-CSF from patients. These autoAbs target at least four non-overlapping conformational epitopes on GM-CSF and are dependent on disulfide bond formation [25]. For anti-IFN-α autoAbs in patients with RA, the neutralizing epitopes were investigated using the phage display random peptide library [14]. The evidence collectively suggested that neutralizing autoAbs in ACAD may be generated via different mechanisms and display distinct characteristics. The results revealed that discontinuous epitopes play major roles in pathogenesis. Herein, inventors of the present disclosure successfully identified the B27 mAb epitope, which was significant in AOID pathogenesis. The phage display technique, in concert with structure-based analysis, presented a promising strategy for the discovery of non-sequential neutralizing epitopes. Lack of IFN-γ-mediated anti-mycobacterial activity due to anti-IFN-γ autoAbs can cause severe symptoms in patients with AOID. Treatments that restore the IFN-γ functions can be useful to the patients. Replacement of the residues at positions 121-127 (SPAAKTG) of human IFN-γ with the corresponding sequence (LPESSLR) from the mouse has been reported to reduce autoAb binding and increase bioactivity of IFN-γ in presence of autoAb [11]. This finding suggested that modification of neutralizing epitopes promotes the escape of IFN-γ from neutralizing autoAbs and enhances IFN-γ-mediated functions. The present disclosure found that B27 epitope is accessed by a population of neutralizing anti-IFN-γ autoAbs. Therefore, modification of the characterized B27 epitope that hinders autoAb interaction would be valuable as a supplement treatment for patients with AOID, who have the autoAbs. Regarding BLAST alignment, the B27 epitope was highly conserved across vertebrates with more than 80% homology. Genetically engineered amino acid variants that retain the immunological activity while reducing reactivity towards autoAbs will be potential candidates for therapeutic approach in AOID. Since autoAbs are diverse across individuals, identification of neutralizing epitopes for anti-IFN-γ autoAbs would provide precise diagnosis and treatment. The present disclosure provided data regarding the epitope recognized by B27 mAb, which was competed for by autoAbs from patients with AOID.

The present disclosure may be embodied in other specific forms without departing from its structures, methods, or other essential characteristics as broadly described herein and claimed hereinafter. The described embodiments are to be considered in all respects only as illustrative, and not restrictive. The scope of the disclosure is, therefore, indicated by the appended claims, rather than by the foregoing description. All changes that come within the meaning and range of equivalency of the claims are to be embraced within their scope.

1. A modified protein of an interferon gamma (IFN-γ) comprising at least one modification of an amino acid located at a position of 27 to 40 of a sequence as setting forth in SEQ ID NO. 1, wherein the modified IFN-γ is capable of initiating cellular signalling towards an immune response upon being administered to a subject.

2. The modified protein of claim 1, wherein the modification alters a discontinuous epitope of the formed IFN-γ.

3. The modified protein of claim 2, wherein the modification comprises substituting at least one of amino acids located at position 27, position 29, and position 30.

4. The modified protein of claim 3, wherein the at least one of amino acids located at position 27, position 29, and position 30 is substituted by alanine, aspartic acid, histidine, proline or tryptophan.

5. The modified protein of claim 3, wherein the formed IFN-γ is resistant against at least one autoantibody capable of neutralizing a wild type IFN-γ at the discontinuous epitope.

6. A method of initiating a cellular signalling towards an immune response in a subject of adult-onset immunodeficiency (AOID) comprising the step of administering a plurality of modified proteins, wherein the modified protein is a modified interferon gamma protein comprising at least one modification of an amino acid located at a position of 27 to 40 of a sequence as setting forth in SEQ ID NO. 1.

7. The method of claim 6, wherein the modification comprises substituting at least one of amino acids located at position 27, position 29, and position 30.

8. The method of claim 7, wherein the at least one of amino acids located at position 27, position 29, and position 30 is substituted by alanine, aspartic acid, histidine, proline, or tryptophan.

9. The method of claim 6, wherein the modification alters a discontinuous epitope of the formed IFN-γ rendering the formed IFN-γ resistant against at least one autoantibody capable of neutralizing a wild type IFN-γ at the discontinuous epitope.

10. A vector comprising a polynucleotide sequence being translatable to produce a modified IFN-γ protein comprising at least one modification of an amino acid located at a position of 27 to 40 of a sequence as setting forth in SEQ ID NO. 1, wherein the modified IFN-γ is capable of initiating cellular signalling towards an immune response upon being administered to a subject.

11. The vector of claim 10, wherein the modification comprises substituting at least one of amino acids located at position 27, position 29, and position 30.

12. The vector of claim 11, wherein the at least one of amino acids located at position 27, position 29, and position 30 is substituted by alanine, aspartic acid, histidine, proline, or tryptophan.

13. The vector of claim 10 is pET-21a, or pQE-10.
